(19) United States
(12) Patent Application Publication
(10) Pub. No.: US 2016/0066952 A1
(43) Pub. Date: Mar. 10, 2016

(54) INSERTION TOOL AND PUNCTURE APPARATUS

(71) Applicant: TERUMO KABUSHIKI KAISHA, Tokyo (JP)

(72) Inventors: Masakatsu KAWAURA, Sunnyvale, CA (US); Nao YOKOI, Sunnyvale, CA (US); Shigeki ARIURA, Ebina-shi (JP)

(73) Assignee: TERUMO KABUSHIKI KAISHA, Tokyo (JP)

(21) Appl. No.: 14/870,743

(22) PCT Filed: Apr. 1, 2013
(86) PCT No.: PCT/JP2013/059894

§ 371 Date: Sep. 30, 2015

(51) Int. Cl. A61B 17/34; A61B 17/04; A61B 17/06

(52) U.S. Cl. CPC A61B 17/3468; A61B 17/06109; A61B 17/0482; A61B 17/3403; A61B 2017/00805

(57) ABSTRACT

An insertion tool is disclosed, which includes a urethral insertion portion of a longitudinal shape configured to be inserted into a urethra, and a vaginal insertion portion of a longitudinal shape configured to be inserted into a vagina. A distal end portion of the vaginal insertion portion is inclined with respect to the urethral insertion portion in a direction in which the distal end portion of the vaginal insertion portion is spaced away from the urethral insertion portion.

### CROSS-REFERENCES TO RELATED APPLICATIONS

This application is a continuation of International Application No. PCT/JP2013/059894 filed on Apr. 1, 2013, the entire content of which is incorporated herein by reference.

### TECHNICAL FIELD

The present disclosure generally relates to an insertion tool and a puncture apparatus.

### BACKGROUND DISCUSSION

If a person suffers from urinary incontinence, for example, from stress urinary incontinence, then urine leakage occurs when abdominal pressure is applied during normal movement or by laughing, coughing, sneezing or the like. This is caused, for example, by a fact that pelvic floor muscles which are muscles supporting the urethra are loosened by birth.

For the treatment of urinary incontinence, surgical therapy is effective, and for example, a living body tissue supporting strip indwelling called “sling” is used. A sling is indwelled in the body to support the urethra (for example, refer to Japanese Patent Laid-Open No. 2010-99499). In order to indwell the sling in the body, the operator would incise the vagina with a scalpel, peel off a region between the urethra and the vagina and cause the peeled off region and the outside to communicate with each other through an obturator foramen using a puncture needle or the like. Then, in such a state as just described, the sling is indwelled in the body.

However, if the vagina is incised, the sling may be exposed to the inside of the vagina through a wound caused by the incision or that such complications as infection from the wound may be caused. Further, since the vagina is incised, there is a drawback that the invasion can be relatively significant and the burden on the patient can be relatively heavy. Further, the urethra may be damaged during the manipulation by the operator, and the finger of operator may be damaged.

### SUMMARY

An insertion tool and a puncture apparatus are disclosed by which a wide distance can be relatively assured between the urethral wall and the vaginal wall while the burden on a living body can be reduced.

An insertion tool is disclosed, which can include a urethral insertion portion of a longitudinal shape configured to be inserted into a urethra, and a vaginal insertion portion of a longitudinal shape configured to be inserted into a vagina, wherein a distal end portion of the vaginal insertion portion is inclined with respect to the urethral insertion portion in a direction in which the distal end portion of the vaginal insertion portion is spaced away from the urethral insertion portion.

According to an exemplary embodiment, preferably, an extension line of the distal end portion and the urethra insertion portion cross with each other.

According to an exemplary embodiment, preferably, an extension line of the distal end portion and the urethral insertion portion do not cross with each other.

According to an exemplary embodiment, preferably, the distal end portion is inclined by 10 to 45 degrees with respect to the urethral insertion portion.

According to an exemplary embodiment, preferably, the distal end portion is spaced away from the urethral insertion portion in an opposite direction in a direction perpendicular to a long axis of the urethral insertion portion.

According to an exemplary embodiment, preferably, the distal end portion has a flattened shape crushed or compressed in an array direction of the urethral insertion portion and the vaginal insertion portion.

According to an exemplary embodiment, preferably, at least one suction hole for sucking a portion of a vaginal wall on the urethra side is provided at the distal end portion.

According to an exemplary embodiment, preferably a region in which the suction hole is formed has a width from 15 to 35 mm.

According to an exemplary embodiment, preferably, the urethral insertion portion has, on a proximal end side thereof, a urethral non-insertion portion which is not inserted into the urethra, the vaginal insertion portion has, on a proximal end side thereof, a vaginal non-insertion portion which is not inserted into the vagina, and the urethral non-insertion portion and the vaginal non-insertion portion are spaced from each other.

According to an exemplary embodiment, preferably, the insertion tool can include a supporting portion configured to support the urethral insertion portion and the vaginal insertion portion, and the supporting portion is spaced away from a body surface in a mounted state in which the urethral insertion portion is inserted in the urethra and the vaginal insertion portion is inserted in the vagina.

According to another aspect of the present disclosure, a puncture apparatus including an insertion tool and a puncture needle is disclosed. The insertion tool can include a urethral insertion portion of a longitudinal shape configured to be inserted into a urethra, and a vaginal insertion portion of a longitudinal shape configured to be inserted into a vagina, a distal end portion of the vaginal insertion portion being inclined with respect to the urethral insertion portion in a direction in which the distal end portion of the vaginal insertion portion is spaced away from the urethral insertion portion. The puncture needle is capable of turning with respect to the insertion tool. When the puncture needle is turned to puncture a living body tissue, a needle tip of the puncture needle passes between the urethral insertion portion and the vaginal insertion portion.

A method is disclosed of forming a path in living body tissue, the method comprising: inserting a urethral insertion portion of a longitudinal shape into a urethra; inserting a vaginal insertion portion of a longitudinal shape into a vagina, wherein a distal end portion of the vaginal insertion portion being inclined with respect to the urethral insertion portion in a direction in which the distal end portion of the vaginal insertion portion is spaced away from the urethral insertion portion; and puncturing a living body tissue with a puncture needle by passing a needle tip of the puncture needle between the urethral insertion portion and the vaginal insertion portion.

With the insertion tool of the present disclosure, the distal end portion of the vaginal insertion portion is inclined with respect to the urethral insertion portion in the direction in which the distal end portion is spaced away from the urethral insertion portion. Therefore, the positional relationship between the urethral insertion portion and the vaginal insertion portion can be made closer to the positional relationship between the urethra and the vagina. Consequently, in the mounted state in which the urethral insertion portion is inserted in the urethra and the vaginal insertion portion is inserted in the vagina, unnatural deformation and so forth of the urethra and the vagina can be prevented, resulting in reduction of the burden on the patient. Further, since the vaginal wall can be spaced away from the urethral wall along the vaginal insertion portion, in the mounted state, the urethral wall and the vaginal wall can be spaced widely from each other. Therefore, with the puncture apparatus of the present disclosure, the contact between the needle tip of the puncture needle and the urethral wall or the vaginal wall can be suppressed effectively and the manipulation can be carried out safely.

### BRIEF DESCRIPTION OF THE DRAWINGS

FIG. 1 is a perspective view depicting a puncture apparatus according to a first embodiment of the present disclosure;

FIG. 2 is a lateral view of the puncture apparatus depicted in FIG. 1;

FIG. 3 is a top plan view depicting a puncture member, which includes the puncture apparatus as depicted in FIG. 1;

FIG. 4 is a top plan view (rear view) depicting a guide portion of a frame provided on the puncture apparatus depicted in FIG. 1;

FIG. 5 is a top plan view (rear view) depicting the guide portion of the frame provided on the puncture apparatus depicted in FIG. 1;

FIG. 6 is a top plan view depicting a fixing portion of the frame provided on the puncture apparatus depicted in FIG. 1;

FIG. 7 is a lateral view of an insertion tool, which includes the puncture apparatus as depicted in FIG. 1;

FIGS. 8A and 8B are a lateral view and a front view, respectively, illustrating a positional relationship between a puncture needle and an obturator foramen (pelvis);

FIG. 9 is a partial enlarged view of a vaginal insertion member, which includes the insertion tool as depicted in FIG. 7;

FIGS. 10A and 10B are cross sectional views illustrating an example of a shape of a vaginal wall in a natural state and a state in which a vaginal insertion portion is inserted in a vagina;

FIG. 11 is a view depicting an implant for use together with the puncture apparatus depicted in FIG. 1;

FIGS. 12A and 12B are views illustrating an operation procedure of the puncture apparatus depicted in FIG. 1;

FIGS. 13A and 13B are views illustrating an operation procedure of the puncture apparatus depicted in FIG. 1;

FIG. 14 is a lateral view illustrating a relationship between the puncture apparatus and the pelvis in the state depicted in FIG. 13A;

FIGS. 15A and 15B are views illustrating an operation procedure of the puncture apparatus depicted in FIG. 1;

FIG. 16 is a lateral view illustrating a relationship between the puncture apparatus and the pelvis in the state depicted in FIG. 15A;

FIGS. 17A and 17B are views illustrating another operation procedure of the puncture apparatus depicted in FIG. 1;

FIG. 18 is a lateral view of an insertion tool which includes a puncture apparatus according to a second exemplary embodiment of the present disclosure; and

FIG. 19 is a cross sectional view of an insertion tool which includes a puncture apparatus according to a third exemplary embodiment of the present disclosure.

### DETAILED DESCRIPTION

In the following, an insertion tool and a puncture apparatus of the present disclosure are described in detail on the basis of preferred embodiments thereof depicted in the accompanying drawings.

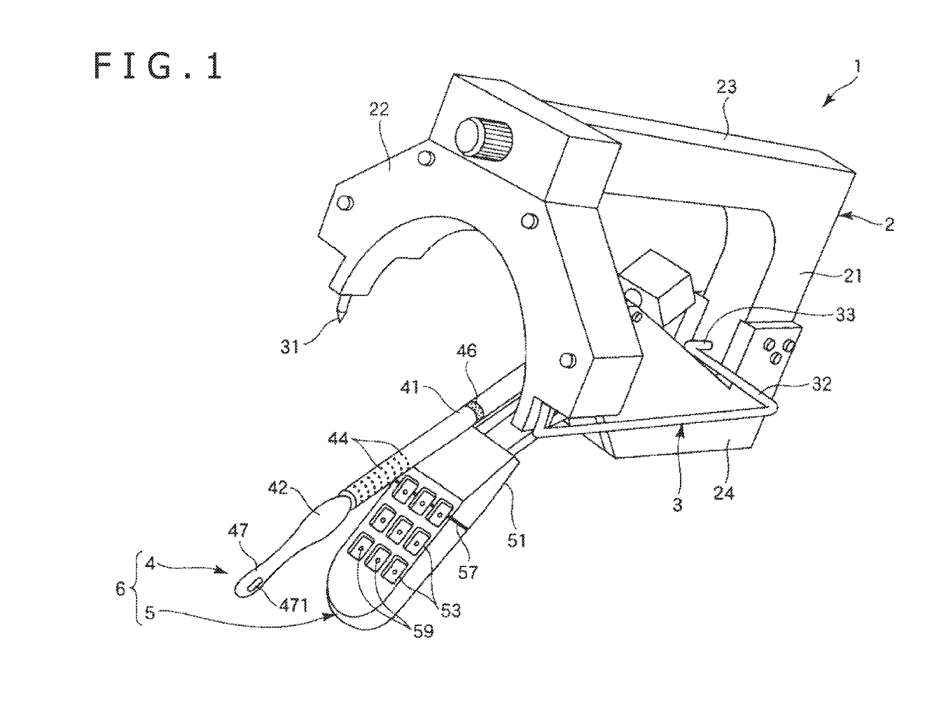

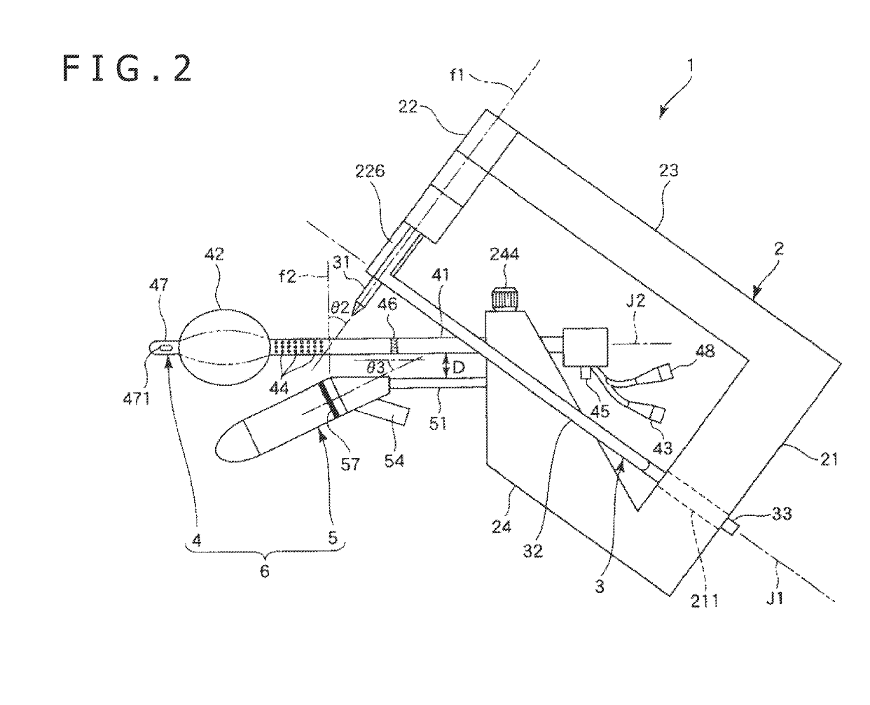

Note that, in the following description, the left side in FIG. 2 is referred to as “distal end,” the right side as “proximal end,” the upper side as “upper,” and the lower side as “lower” for the convenience of description. FIGS. 1 and 2 depict the puncture apparatus in a state in which the puncture apparatus is not used as yet, and in the following description, this state is referred to also as “initial state” for the convenience of description. Further, a state in which the puncture apparatus (insertion tool) depicted in FIG. 1 is mounted on a patient is referred to also as “mounted state.”

The puncture apparatus 1 depicted in FIGS. 1 and 2 is a device used for the treatment of, for example, female urinary incontinence, for example, for embedding a living body tissue supporting indwelling for the treatment of urinary incontinence into a living body.

The puncture apparatus 1 can include a frame (supporting member) 2, a puncture member 3, a urethral insertion member 4, and a vaginal insertion member 5. The puncture member 3, urethral insertion member 4 and vaginal insertion member 5 are supported on the frame 2. Further, in the puncture apparatus 1, the urethral insertion member 4 and the vaginal insertion member 5 configure an insertion tool 6. In the following, the frame 2, puncture member 3 and insertion tool 6 are described in order.

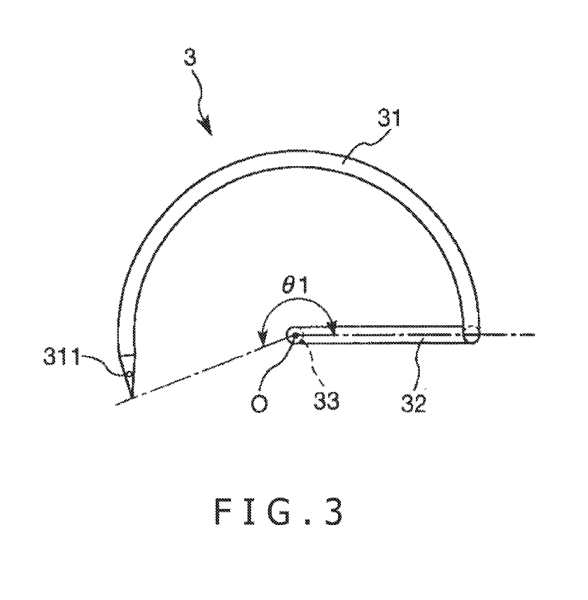

As depicted in FIGS. 1 to 3, the puncture member 3 has a puncture needle 31 for puncturing a living body tissue, a shaft portion 33, and a connection portion 32 for connecting the puncture needle 31 and the shaft portion 33 to each other. Note that the puncture needle 31, connection portion 32 and shaft portion 33 may be formed integrally, or at least one of them may be formed as a separate member from the other members. The puncture member 3 is configured such that it punctures a living body tissue between the urethra and the vagina from the inguinal region (region different from the urethra and the vagina).

The puncture needle 31 can have an arcuate shape. The puncture needle 31 can have a central angle θ1, which is not limited specifically but is set suitably in accordance with various conditions. However, as hereinafter described, the central angle θ1 is set such that the puncture needle 31 can enter the body from one of the inguinal regions of a patient, pass between the urethra and the vagina and project to the outside of the body from the other inguinal region. In accordance with an exemplary embodiment, for example, the central angle θ1 preferably is 150 to 270 degrees, more preferably is 170 to 250 degrees, and most preferably is 190 to 230 degrees.

A through-hole 311 for holding an implant 9 hereinafter described is formed at a distal end portion of the puncture needle 31.

The shaft portion 33 extends along an axis J1 which crosses with the center O of the puncture needle 31 and crosses orthogonally with a plane f1 which can include the puncture needle 31.

The connection portion 32 connects the proximal end of the puncture needle 31 and the distal end of the shaft portion 33. Further, the connection portion 32 exhibits a substantially L shape bent substantially at the right angle in the middle thereof. The connection portion 32 can function also as a gripping portion to be gripped by an operator when the operator operates the puncture member 3.

The constituent material of the puncture member 3 is not limited especially, but, for example, various metal materials such as stainless steel, aluminum or aluminum alloy, and titanium or titanium alloy can be used.

The frame 2 supports the puncture member 3 for turning motion and allows the urethral insertion member 4 and the vaginal insertion member 5 to be removably fixed thereto. The frame 2 has a function of determining a puncture path of the needle tip of the puncture needle 31 when the puncture needle 31 punctures a living body tissue. In accordance with an exemplary embodiment, for example, the frame 2 can determine the positional relationship among the puncture member 3, urethral insertion member 4 and vaginal insertion member 5 such that, when the puncture needle 31 punctures the living body tissue, the needle tip of the puncture needle 31 may pass the region between the urethral insertion member 4 and the vaginal insertion member 5 without interfering with any of the urethral insertion member 4 and the vaginal insertion member 5. Consequently, the puncture needle 31 can puncture the living body tissue keeping away the urethra and the vaginal wall with certainty.

As depicted in FIGS. 1 and 2, the frame 2 has a bearing portion 21, which can support the shaft portion 33 of the puncture member 3 for rotation thereon, a guide portion 22 which covers the puncture needle 31 and guides the needle tip of the puncture needle 31, a connection portion 23 which connects the bearing portion 21 and the guide portion 22 to each other, and a fixing portion 24 to which the urethral insertion member 4 and the vaginal insertion member 5 are fixed.

The bearing portion 21 is positioned on the proximal end side of the puncture apparatus 1 and extends in a direction substantially orthogonal to the axis J1. A through-hole 211 is formed on the axis J1 of the bearing portion 21, and the shaft portion 33 is inserted for rotation in the through-hole 211. Consequently, the puncture member 3 is supported for turning motion around the axis J1 on the frame 2.

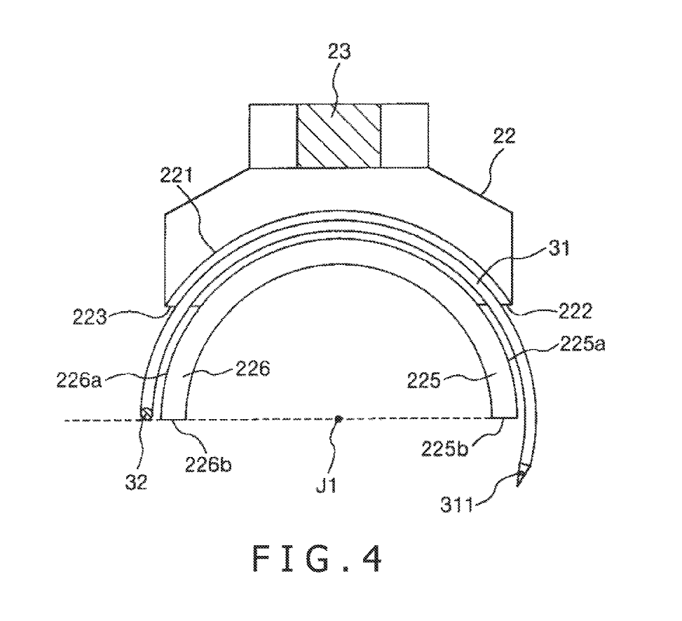

The guide portion 22 is positioned on the distal end side of the puncture apparatus 1 and disposed in an opposing relationship to the bearing portion 21. As depicted in FIG. 4, an arcuate guide groove 221 is formed in the guide portion 22 such that it accommodates and guides the puncture needle 31 therein. By the guide groove 221, puncture of the puncture needle 31 can be carried out relatively accurately. In accordance with an exemplary embodiment, the overall length of the guide groove 221 is smaller than the overall length of the puncture needle 31, and in an initial state, the needle tip of the puncture needle 31 projects from one end 222 of the guide groove 221 while a proximal end portion of the puncture needle 31 projects from the other end 223.

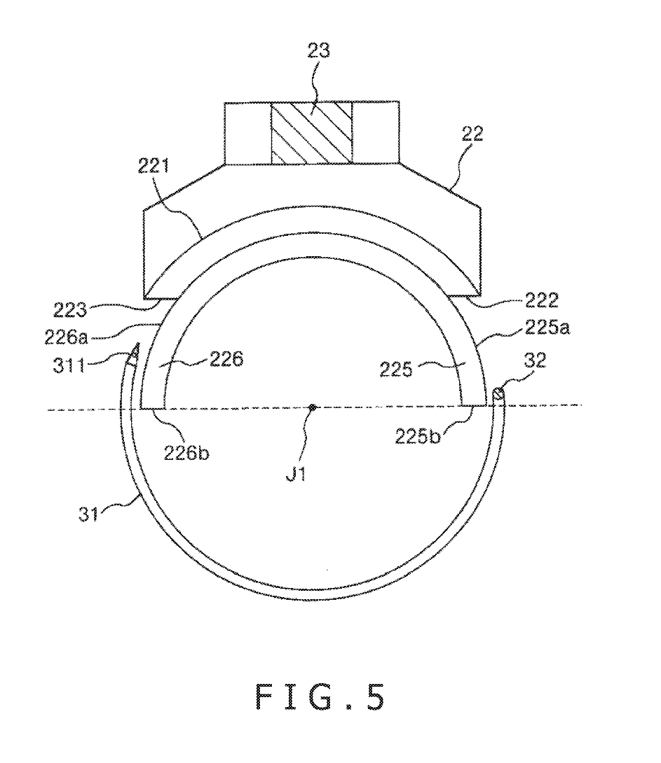

Further, at a portion of the guide portion 22 which is positioned on the inner side with respect to the guide groove 221 (puncture needle 31), a pair of projections 225 and 226 is formed such that they project to the lower side. Of the projections 225 and 226, the projection 225 extends in an arc along the circumference of a distal end portion of the puncture needle 31 while the other projection 226 extends in an arc along the circumference of a proximal end portion of the puncture needle 31. Further, outer side faces 225a and 226a of the projections 225 and 226 are configured from faces continuing to the guide groove 221. In an initial state, the needle tip of the puncture needle 31 is positioned on the lower side with respect to a lower face 225b of the projection 225 while the proximal end of the puncture needle 31 is positioned at a substantially same height as a lower face 226b of the projection 226. If the puncture needle 31 is turned in the clockwise direction in FIG. 4 from the initial state depicted in FIG. 4, then the puncture needle 31 further projects from the guide groove 221 to be brought into a state depicted in FIG. 5. Note that, depending upon the central angle θ1 of the puncture needle 31, the length of the projection 225 and so forth, the needle tip of the puncture needle 31 may be positioned at the same height as the lower face 225b of the projection 225 or may be positioned on the upper side with respect to the lower face 225b.

In accordance with an exemplary embodiment, the projections 225 and 226 function as pressing portions for pressing the surface of a living body. For example, if the patient has a comparatively thick subcutaneous tissue, then by pressing the projections 225 and 226 against the living body surface, the projections 225 and 226 can be sunk into the living body to allow the puncture needle 31 to puncture the living body deeply. Therefore, the puncture apparatus 1 can cope with a patient whose subcutaneous tissue has a comparatively large thickness.

By forming the projections 225 and 226 on the inner side with respect to the guide groove 221 as in the present embodiment, the puncture needle 31 can be prevented from being hidden by the projections 225 and 226. Therefore, as hereinafter described, it becomes relatively easy to carry out a step for threading a string 91 of the implant 9 into the through-hole 311 of the puncture needle 31, resulting in improvement of the operability of the puncture apparatus 1.

The connection portion 23 connects the bearing portion 21 and the guide portion 22 to each other. Further, the connection portion 32 is in the form of a bar extending substantially in parallel to the axis J1. The connection portion 32 can function also as a gripping portion, and an operator can grip the connection portion 32 to use the puncture apparatus 1.

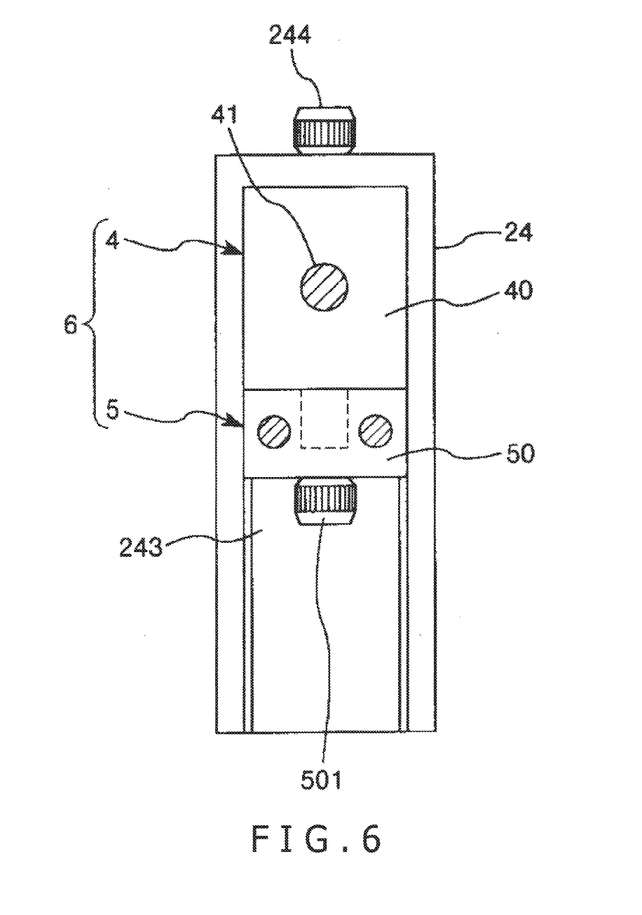

The fixing portion 24 is disposed in an opposing relationship to the connection portion 23 with the axis J1 interposed therebetween. As depicted in FIG. 6, the fixing portion 24 has a recessed portion 243 into which a supporting portion 60 hereinafter described of the insertion tool 6 is to be fitted, and a male thread 244. If the supporting portion 60 is fitted into the recessed portion 243 and besides the male thread 244 is tightened into a female thread (not depicted) of the supporting portion 60, then the insertion tool 6 can be fixed to the fixing portion 24. Note that the configuration of the fixing portion 24 is not limited especially only if the urethral insertion member 4 and the vaginal insertion member 5 can be fixed to the fixing portion 24.

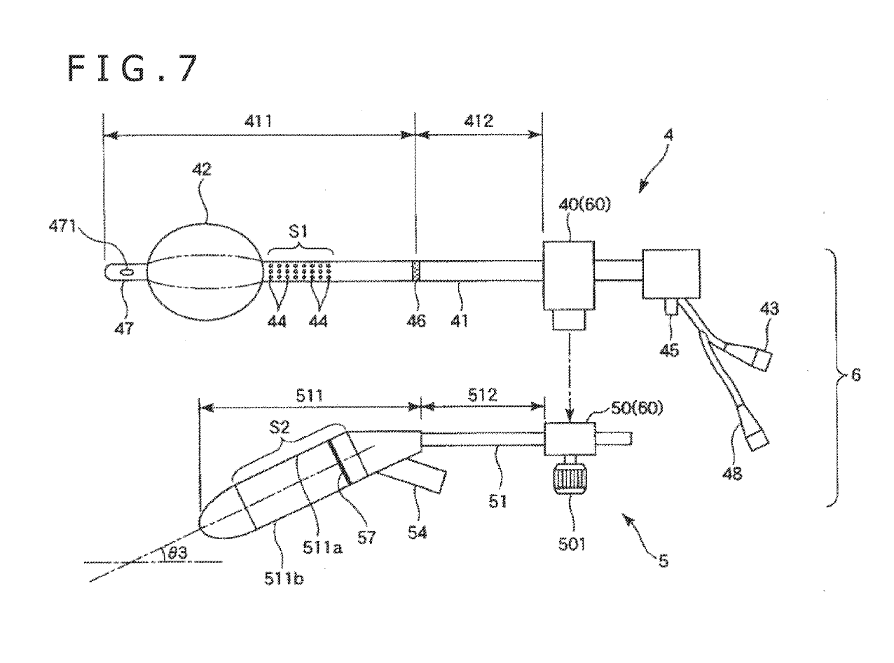

As depicted in FIGS. 1 and 7, the insertion tool 6 has a urethral insertion portion 41 which is to be inserted into the urethra, a vaginal insertion portion 51 which is to be inserted into the vagina, and the supporting portion 60 which supports the urethral insertion portion 41 and the vaginal insertion portion 51 thereon. As described hereinabove, the insertion tool 6 is configured from the urethral insertion member 4 and the vaginal insertion member 5, and the urethral insertion member 4 can include the urethral insertion portion 41 while the vaginal insertion member 5 can include the vaginal insertion portion 51. Further, the supporting portion 60 has a supporting portion 40, which is provided on the urethral insertion member 4 and supports the urethral insertion portion 41 and a supporting portion 50, which is provided on the vaginal insertion member 5 and supports the vaginal insertion portion 51. In the insertion tool 6, the urethral insertion member 4 and the vaginal insertion member 5 can be removably mounted through the supporting portions 40 and 50, respectively. In the following, the urethral insertion member 4 and the vaginal insertion member 5 are described in order.

The urethral insertion member 4 has the elongate urethral insertion portion 41, which is inserted to the middle into the urethra, and the supporting portion 40, which supports the urethral insertion portion 41. Note that, in the following description, a portion of the urethral insertion portion 41 which is positioned in the urethra (including the bladder) in the mounted state of the puncture apparatus 1 is referred to also as “insertion portion 411” and another portion of the urethral insertion portion 41 which is exposed to the outside of the body from the urethral orifice in the mounted state and extends to the supporting portion 40 is referred to also as “non-insertion portion (urethral non-insertion portion) 412” for the convenience of description.

The urethral insertion portion 41 has a form of a linear pipe rounded at a distal end thereof. Further, the urethral insertion portion 41 has an inflatable/deflatable balloon 42 and a urine discharge portion 47 provided at a distal end portion of the insertion portion 411 thereof. The balloon 42 can function as a regulation portion for regulating the position of the urethral insertion member 4 in the axial direction in the urethra. In accordance with an exemplary embodiment, for example, when the puncture apparatus 1 is used, the balloon 42 is inserted into the bladder of the patient and then inflated. Then, the inflated balloon 42 is caught by the bladder neck thereby to fix the position of the urethral insertion member 4 with respect to the bladder and the urethra. Meanwhile, the urine discharge portion 47 is used to discharge urine in the bladder therethrough.

The balloon 42 is connected to a balloon port 43 disposed at a proximal end portion of the urethral insertion portion 41 passing through the inside of the urethral insertion portion 41. A balloon inflation tool such as a syringe can be connected to the balloon port 43. If working fluid (liquid such as saline solution, gas or the like) is supplied from the balloon inflation tool into the balloon 42, then the balloon 42 is inflated, but if the working fluid is taken out from the balloon 42 by the balloon inflation tool conversely, then the balloon 42 is deflated. Note that, in FIG. 7, the balloon 42 in a deflated state is indicated by an alternate long and two short dashes line while the balloon 42 in an inflated state is indicated by a solid line.

A discharge hole 471 is provided in the urine discharge portion 47 such that the discharge hole 471 communicates the inside and the outside of the urine discharge portion 47 with each other. Further, the urine discharge portion 47 passes through the inside of the urethral insertion portion 41 and is connected to a urine discharge port 48 provided at a proximal end portion of the urethral insertion portion 41. Therefore, urine introduced from the discharge hole 471 can be discharged from the urine discharge port 48.

The balloon 42 and the urine discharge portion 47 can be configured, for example, from a double lumen.

Further, a plurality of suction holes 44 are formed at an intermediate portion of the insertion portion 411. The plurality of suction holes 44 are disposed over an overall area of the urethral insertion portion 41 in a circumferential direction. Each of the suction holes 44 extends through the inside of the urethral insertion portion 41 and is connected to a suction port 45 provided at a proximal end portion of the urethral insertion portion 41. A suction apparatus such as a pump can be connected to the suction port 45. If the suction apparatus is rendered operative in a state in which the urethral insertion portion 41 is inserted in the urethra, then the urethral wall can be sucked and fixed to the urethral insertion portion 41. Then, if the urethral insertion portion 41 in this state is pushed to the distal end side (into the body), then also the urethra is pushed in together, and for example, the bladder can be displaced to a position which does not overlap with the puncture passage of the puncture member 3 and the puncture passage of the puncture member 3 can be assured. Therefore, the puncture of the puncture member 3 can be carried out relatively accurately and safely. Note that the number of suction holes 44 is not limited particularly and may be, for example, one. Further, the disposition of the suction holes 44 is not limited particularly, and the suction holes 44 may be formed, for example, at only part of the urethral insertion portion 41 in the circumferential direction.

Further, a marker 46 for allowing confirmation of the insertion depth of the urethral insertion portion 41 in the urethra is provided at a boundary portion between the insertion portion 411 and the non-insertion portion 412. The marker 46 is positioned at the urethral orifice when the urethral insertion portion 41 is inserted into the urethra until the balloon 42 is positioned in the bladder. Consequently, the insertion depth of the insertion portion 411 in the urethra can be relatively easily confirmed. As the marker 46, it is only necessary that it be visually recognized from the outside, and the marker 46 can be configured, for example, from a colored portion, a recessed or projected portion or the like. Note that graduations representing the distances from the distal end of the urethral insertion portion 41 may be applied in place of the marker 46.

The length of the insertion portion 411 is not limited particularly and can be set suitably depending upon the length of the urethra, the shape of the bladder and so forth of the patient. Since the length of the urethra of general women is approximately 30 to 50 mm, the length of the insertion portion 411 preferably is approximately 50 to 100 mm.

Although the length of the non-insertion portion 412 (spacing distance between the urethral orifice and the supporting portion 40) is not limited particularly, it preferably is equal to or shorter than approximately 100 mm, and more preferably is approximately 20 to 50 mm. Consequently, the non-insertion portion 412 can be made with an appropriate length and is improved in operability. For example, if the length of the non-insertion portion 412 exceeds the upper limit value given above, then depending upon the configuration of the frame 2 and so forth, the center of gravity of the puncture apparatus 1 is displaced by a great distance from the patient, resulting in that the stability of the puncture apparatus 1 in the mounted state may be deteriorated.

The constituent material of the urethral insertion member 4 is not limited particularly, and for example, various metal materials such as stainless steel, aluminum or aluminum alloy, titanium or titanium alloy and so forth or various resin materials can be used.

Here, the inclination angle θ2 (the angle defined by the axis J1 and the axis J2) of the plane f1 with respect to a plane f2 orthogonal to the axis J2 of the urethral insertion portion 41 preferably is approximately 20 to 60 degrees, more preferably is approximately 30 to 45 degrees, and most preferably is approximately 35 to 40 degrees. In accordance with an exemplary embodiment, for example, the angle defined by the plane f1 and a plane orthogonal to the axis of the urethra preferably is approximately 20 to 60 degrees, more preferably is approximately 30 to 45 degrees, and most preferably is approximately 35 to 40 degrees. By this, the puncture of the puncture member 3 can be carried out relatively easily and the puncture distance by the puncture member 3 can be made shorter.

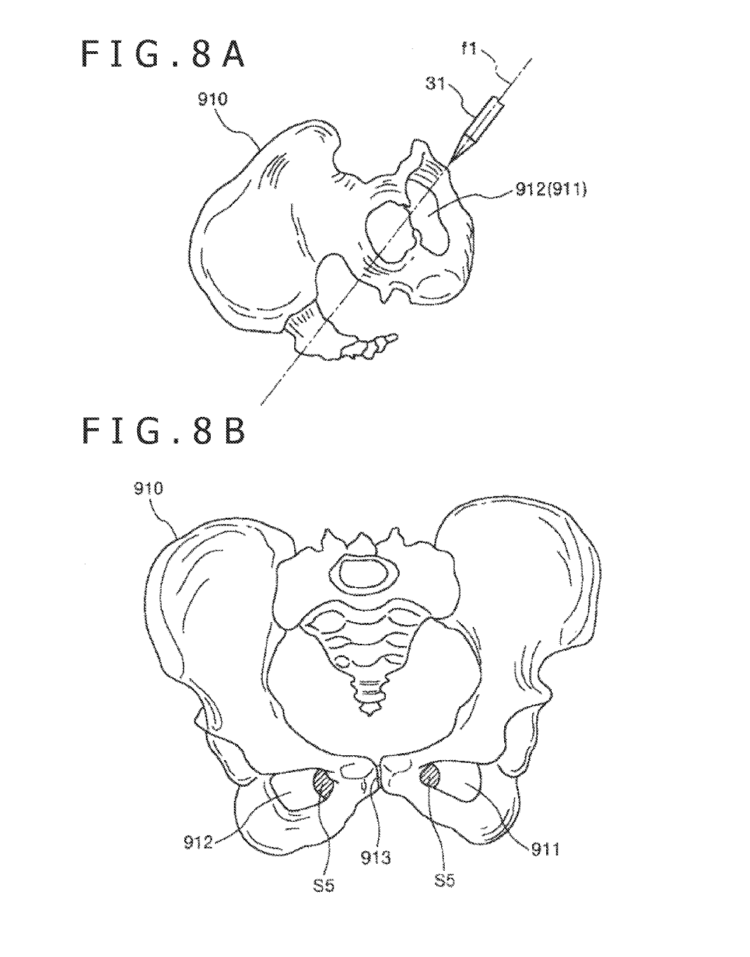

Describing more particularly, by setting the inclination angle θ2 within the range described above, the puncture needle 31 can capture left and right obturator foramens 911 and 912 of a pelvis 910 widely in plane and the puncture space of the puncture needle 31 can be assured wide as depicted in FIG. 8A. In accordance with an exemplary embodiment, for example, in a state in which the patient is placed in a predetermined position (lithotomy position), the puncture needle 31 can puncture in a comparatively vertical direction with respect to the obturator foramens 911 and 912. Therefore, the puncture of the puncture needle 31 can be carried out relatively easily. In addition, where the puncture needle 31 punctures in a comparatively vertical direction with respect to the obturator foramens 911 and 912, it passes a shallow portion of the tissue, and therefore, the needle tip of the puncture needle 31 can pass over a shorter distance between the left and right obturator foramens 911 and 912. Therefore, as depicted in FIG. 8B, the puncture needle 31 can pass the obturator foramens 911 and 912 rather near to a pubic symphysis 913, preferably a safety zone S5. Since the safety zone S5 is a region, which can include a comparatively small number of nerves and blood vessels to which damage is to be avoided, the puncture needle 31 can puncture in safe. Therefore, the invasion can be reduced and the burden on the patient can be reduced. By setting the inclination angle θ2 to such a range as described above, the puncture of the patient by the puncture needle 31 can be appropriately carried out. Further, by the puncture at the angle specified as above, the tissue between the mid urethra, which refers to a middle portion of the urethra in the lengthwise direction and the vagina can be determined as a target. The position between the mid urethra and the vagina is a position suitable as a region into which the implant 9 is to be embedded to carry out the treatment of urinary incontinence.

In contrast, if the inclination angle θ2 is smaller than the lower limit value given hereinabove or is greater than the upper limit value given hereinabove, then depending upon the individual differences of patients, the posture during the procedure, the puncture needle 31 may not capture the obturator foramens 911 and 912 widely in plane or may fail to sufficiently shorten the puncture passage.

In accordance with an exemplary embodiment, for example, the puncture needle 31 can easily puncture the region between the mid urethra and the vagina if it punctures in a state in which the position of the region between the mid urethra and the vagina is displaced such that the urethra, the vagina or both of the urethra and the vagina are pushed into the inner side of the body. The method of pushing one of the urethra and the vagina to the inner side of the body can be, for example, a method of placing the urethral insertion member 4 and/or the vaginal insertion member 5 into a state in which the urethral insertion member 4 and/or the vaginal insertion member 5 are inserted to appropriate positions, absorbing the urethra and/or the vagina through suction holes 44 and 59 (described hereinafter) provided in the urethral insertion member 4 and/or the vaginal insertion member 5 and then moving the urethral insertion member 4 and/or the vaginal insertion member 5 further to the inner side of the body at predetermined positions along the individual axial lines thereof. In such a manner, in a state in which the position of the puncture needle 31 is displaced such that at least one of the urethra and the vagina is pushed into the inner side of the body, by causing the puncture member 3 to vertically puncture the left and right obturator foramens 911 and 912 of the pelvis 910, a path can be formed at a position suitable for indwelling of the implant 9.

In accordance with an exemplary embodiment, for example, it can be preferable to set the locus of the puncture member 3 so as to pass the safety zone S5 of the left and right obturator foramens 911 and 912 of the pelvis 910, displace at least one of the urethra and the vagina to the inner side of the body so that the locus may be positioned between the mid urethra and the vagina and cause the puncture member 3 to puncture along the locus to form a path.

As depicted in FIGS. 1 and 7, the vaginal insertion member 5 has the elongate vaginal insertion portion 51, which is to be inserted partway into the vagina, and the supporting portion 50 which supports the vaginal insertion portion 51. Note that, in the following description, a portion of the vaginal insertion member 5 to be positioned in the vagina in the mounted state is referred to as “insertion portion (distal end portion) 511” and another portion of the vaginal insertion member 5. which is exposed to the outside of the body from the vaginal orifice in the mounted state and extends to the supporting portion 50 is referred to also as “non-insertion portion (vaginal non-insertion portion) 512” for the convenience of description.

The insertion portion 511 has a linear shape. Further, the insertion portion 511 extends in an inclined relationship with respect to the insertion portion 411 such that the distal end side of the insertion portion 511 is spaced away from the insertion portion 411. Further, the insertion portion 511 is disposed such that an extension line thereof crosses with the urethral insertion portion 41. In accordance with an exemplary embodiment, for example, in a top plan view as viewed from above in FIG. 7, the insertion portion 511 is provided such that it extends in a direction as and in an overlapping relationship with the urethral insertion portion 41. Further, the insertion portion 511 is provided in a spaced relationship from the urethral insertion portion 41 in an opposite direction in a direction perpendicular to the long axis of the urethral insertion portion 41 (in the upward and downward direction in FIG. 7).

In such a manner described above, by forming the insertion portion 511 in an inclined relationship with respect to the insertion portion 411, the positional relationship of the insertion portions 411 and 511 can be made closer to the positional relationship between the urethra and the vagina in comparison with those in an alternative case in which the insertion portion 511 is not inclined. Therefore, in the mounted state, the puncture apparatus 1 can be held stably on the patient and the burden on the patient is reduced.

Although the inclination angle θ3 of the insertion portion 511 with respect to the insertion portion 411 is not limited particularly, it preferably is, for example, approximately 10 to 45 degrees, and more preferably is approximately 20 to 30 degrees. By such an inclination angle θ3 as just described, the effect described above can be exhibited more significantly. In accordance with an exemplary embodiment, for example, if the inclination angle θ3 is smaller than the lower limit value described above or is greater than the upper limit value described above, then depending upon the individual differences of patients, the posture during the procedure or the like, the vagina or the urethra may be deformed unnaturally in the mounted state and the puncture apparatus 1 may not be held stably.

Note that the insertion portion 511 may be disposed such that the extension line thereof does not cross with the urethral insertion portion 41. In accordance with an exemplary embodiment, for example, in a plan view as viewed from the upper side in FIG. 7, the insertion portion 511 may be provided such that it extends in a direction different from that of the urethral insertion portion 41.

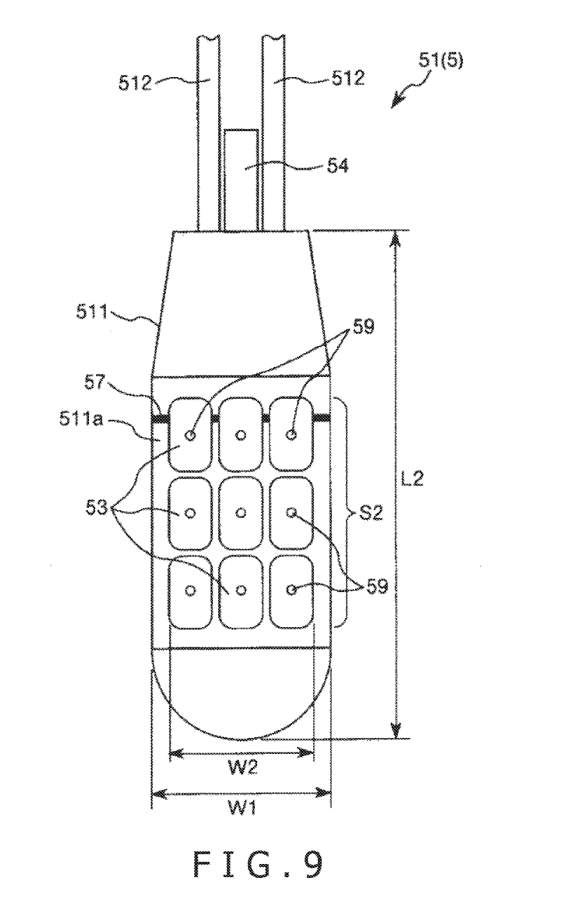

As depicted in FIG. 9, the insertion portion 511 has a flattened shape crushed or compressed n the upward and downward direction of the puncture apparatus 1 (array direction of the urethra and the vagina). Further, the insertion portion 511 has a central portion whose width is substantially fixed and a rounded distal end portion. Although the length L2 of the insertion portion 511 is not limited particularly, for example, it preferably is approximately 20 to 100 mm, and more preferably is approximately 30 to 60 mm. Further, although the width W1 of the insertion portion 511 is not limited particularly, it preferably is approximately 10 to 40 mm, and more preferably is approximately 20 to 30 mm. Further, although the thickness of the insertion portion 511 is not limited particularly, it preferably is approximately 5 to 25 mm, and more preferably is approximately 10 to 20 mm. By forming the insertion portion 511 with such a length×width×thickness as given above, the insertion portion 511 comes to have a shape and a size suitable for general vaginas. Therefore, the stability of the puncture apparatus 1 in the mounted state increases and the burden on the patient can be reduced.

Further, a plurality of bottomed recessed portions 53 are formed on an upper face (face on the urethral insertion portion 41 side) 511a of the insertion portion 511. Note that the number of recessed portions 53 is not limited particularly and may be, for example, one. The bottom surface of each of recessed portions 53 is provided with a suction hole 59. The suction hole 59 is connected to a suction port 54 provided at a proximal end portion of the insertion portion 511 past the inside of the insertion portion 511. The suction port 54 is provided so as to be positioned outside the living body in the mounted state. A suction apparatus such as a pump can be connected to the suction port 54, and if the suction apparatus is rendered operative in a state in which the insertion portion 511 is inserted in the vagina, then the vaginal front wall which is an upper face of the vaginal wall is absorbed and fixed to the insertion portion 511. Then, if the vaginal insertion portion 51 is pushed in to the distal end side (into the body) in a state in which the vaginal wall is absorbed and fixed, then the vaginal wall can be pushed in together with the vaginal insertion portion 51. Therefore, the disposition or the shape of the vaginal wall can be arranged and a puncture passage of the puncture member 3 can be relatively assured, and the puncture of the puncture member 3 can be carried out relatively accurately and safely.

A region S2 in which the plurality of recessed portions 53 are formed is disposed in an opposing relationship to a region S1. Further, the needle tip of the puncture member 3 passes between the regions S1 and S2. Since the urethral rear wall which is a lower face of the urethral wall is absorbed to the insertion portion 411 in the region S1 while the vaginal front wall is absorbed to the insertion portion 511 in the region S2, the urethral wall and the vaginal wall are spaced away from each other by a greater distance between the regions S1 and S2. Therefore, by causing the puncture member 3 to pass through such a region as just described, the puncture member 3 can be caused to puncture with a higher degree of safeness.

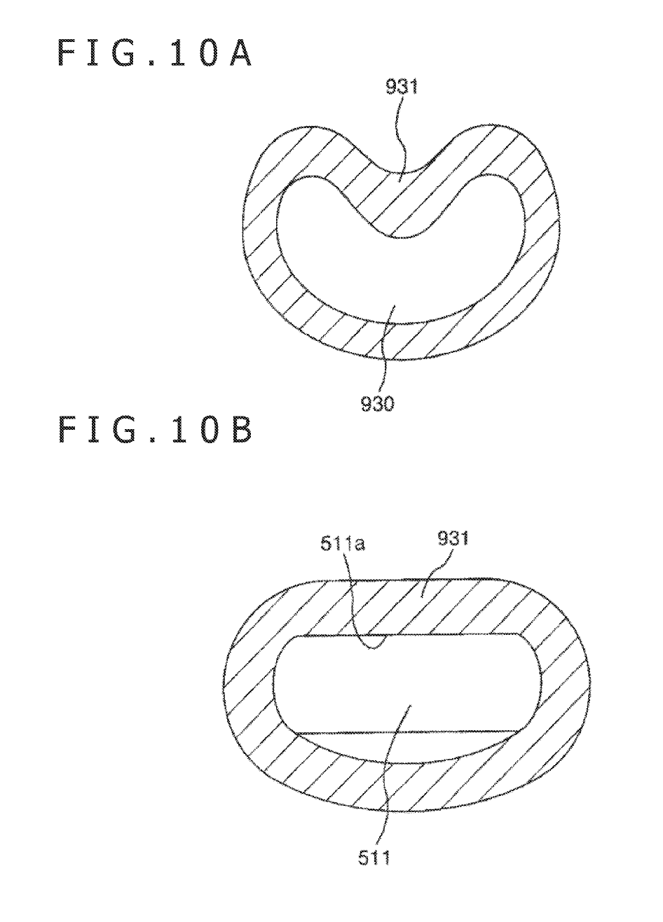

The region S2 spans over a substantially overall area of the upper face 511a in the widthwise direction. Although the width W2 of the region S2 is not limited particularly, it preferably is approximately 9 to 39 mm, and more preferably is approximately 19 to 29 mm. With such a width W2 as just described, the vaginal front wall can be absorbed to the insertion portion 511 with a higher degree of certainty without being influenced much by the shape of the vaginal wall. In accordance with an exemplary embodiment, for example, depending upon a patient, a vagina 930 may be formed such that a vaginal front wall 931 thereof partly hangs into the vagina 930 as depicted in FIG. 10A. In addition, in such a case as just described, if the region S2 has such a width W2 as described above, not only the hanging portion but also portions on the opposite sides of the hanging portion can be absorbed with certainty as depicted in FIG. 10B. Therefore, the vaginal front wall 931 can be spaced away from the urethra with a higher degree of certainty without being influenced by the shape of the vagina 930. In accordance with an exemplary embodiment, for example, in the present embodiment, since the insertion portion 511 has a flattened shape, the vaginal front wall 931 can be absorbed so as to be spaced further away from the urethra, and the living body tissue between the urethral wall and the vaginal wall can be widened further.

Further, a marker (puncture position confirmation portion) 57 can be provided on the insertion portion 511 such that the puncture route of the puncture apparatus 1 can be confirmed therefrom. In accordance with an exemplary embodiment, for example, the puncture apparatus 1 can be secured such that it can puncture a region between the vaginal wall and the urethral wall, which exist on an upper face at a position of the insertion portion 511 at which the marker 57 exists. Therefore, the operability and the safety of the insertion tool 6 can be improved. The marker 57 is provided at least on a lower face 511b of the insertion portion 511. Since the lower face 511b is a face which is directed to the vaginal orifice side in the inserted state and can be confirmed visually by the operator through the vaginal orifice, by providing the marker 57 on the lower face 511b, the puncture route of the puncture apparatus 1 can be confirmed with a higher degree of certainty. In addition, the insertion depth of the insertion portion 511 in the vagina can be confirmed. Note that it is only necessary for the marker 57 to be viewed from the outside, and the marker 57 can be configured, for example, from a colored portion, a recessed or projected portion or the like.

The non-insertion portion 512 has a form of a thin bar extending substantially in parallel to the urethral insertion portion 41. Although the spacing distance D between the non-insertion portion 512 and the urethral insertion portion 41 is not limited particularly, it preferably is approximately 10 to 40 mm in accordance with the spacing distance between the urethral orifice and the vaginal orifice of general women.

Although the length of the non-insertion portion 512 (spacing distance between the vaginal orifice and the supporting portion 50) is not limited particularly, it preferably is equal to or smaller than approximately 100 mm, and more preferably is approximately 20 to 50 mm. This causes the non-insertion portion 512 to have an appropriate length, and the operability is improved. If the length of the non-insertion portion 512 exceeds the upper limit value described above, then depending upon the configuration of the frame 2, the center of gravity of the puncture apparatus 1 is spaced by a great distance from the patient, and the stability of the puncture apparatus 1 in a mounted state may be deteriorated.

The supporting portion 50 has a male thread 501 provided thereon, and the supporting portions 40 and 50 can be fixed to each other by tightening the male thread 501 into a female thread (not depicted) of the supporting portion 40.

The constituent material of the vaginal insertion member 5 is not limited particularly, and, for example, various metal materials such as stainless steel, aluminum or aluminum alloy, titanium or titanium alloy and so forth or various resin materials can be used similarly to the vaginal insertion member 4.

The configuration of the puncture apparatus 1 is such as described above.

Note that, while, in the puncture apparatus 1, the urethral insertion member 4 and the vaginal insertion member 5 which configure the insertion tool 6 are configured removable, the configuration of the urethral insertion member 4 and the vaginal insertion member 5 is not limited to this, and the urethral insertion member 4 and the vaginal insertion member 5 may be configured non-removable.

Further, while the urethral insertion portion 41 in the puncture apparatus 1 is fixed to the supporting portion 40, the configuration of the urethral insertion portion 41 and the supporting portion 40 is not limited to this, and the urethral insertion portion 41 may be configured so as to allow selection between a state in which the urethral insertion portion 41 is fixed to the supporting portion 40 and another state in which the urethral insertion portion 41 is slidably movable in the axial direction on the supporting portion 40. In accordance with an exemplary embodiment, for example, such a configuration may be adopted that, if a male thread provided on the supporting portion 40 is loosened, then the urethral insertion portion 41 is placed into a state in which it can slidably move on the supporting portion 40, but if the male thread is tightened, then the urethral insertion portion 41 is placed into another state in which it is fixed to the supporting portion 40. With the configuration just described, since the length of the non-insertion portion 412 can be adjusted, the convenience in use of the insertion tool 6 is improved. Note that this similarly applies also to the vaginal insertion portion 51.

Further, while, in the puncture apparatus 1, each portion can be fixed to the frame 2 such that the inclination angle θ2 is fixed, the configuration is not limited to this, and the inclination angle θ2 may be variable. If this configuration is applied, then since inclination angle θ2 can be adjusted in accordance with the patient, the convenience in use of the puncture apparatus 1 is further improved.

Further, in order to position the needle tip of a syringe 2000 hereinafter described between the regions S1 and S2, an ultrasonic marker indicative of the position of the region S2 of the insertion portion 511 may be provided. Where the ultrasonic marker is provided, while the operator observes an ultrasonic wave image formed using a transabdominal ultrasonic probe or the like, the operator can position the needle tip of the syringe 2000 between the regions S1 and S2. Therefore, humoral peeling off can be carried out with a relatively higher degree of certainty. The constituent material of the ultrasonic marker is not limited particularly if the ultrasonic marker can be visually confirmed on an ultrasonic wave image. For example, the ultrasonic marker may be configured using various metal materials such as, for example, stainless steel, aluminum or aluminum alloy, and titanium or titanium alloy and may have a plurality of fine recessions and projections on the surface thereof. In accordance with an exemplary embodiment, for example, if a plurality of fine recessions and projections are formed on the surface of a portion configured from a metal material, then the portion at which the recessions and projections are formed can be detected by ultrasonic waves.

In the following, usage of the puncture apparatus 1 is described. However, prior to the description of the usage, the implant 9 for use with the puncture apparatus 1 is described.

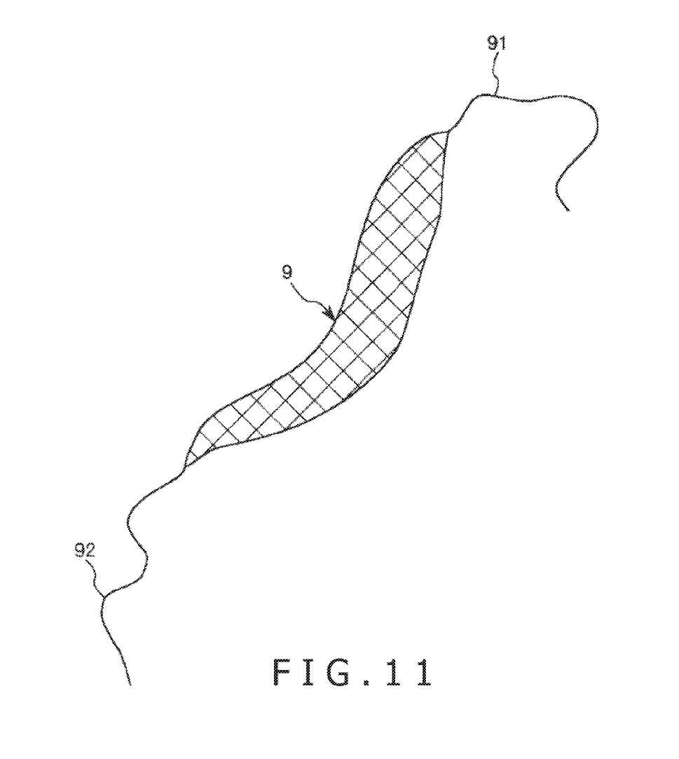

The implant (living body tissue supporting indwelling) 9 depicted in FIG. 11 is a tool which can be embedded for the treatment of urinary incontinence of a female, for example, a tool which supports the urethra, for example, a tool which supports when the urethra tends to move to the vaginal wall side, the urethra so as to restrict the movement of the urethra in a direction away from the vaginal wall. For the implant 9, for example, an elongate article having flexibility can be used.

The implant 9 has a form of a net and has a strip-like general shape. The implant 9 is called “sling.” Note that the implant 9 can be configured from an article formed by crossing linear objects with each other into a braid (lattice shape), for example, a net-like braid. The linear objects may be those having a circular transverse sectional shape, or those having a flattened transverse sectional shape, for example, those of strip-like shape (ribbon shape). To one end of the implant 9, one end portion of the string 91 is fixed, and to the other end portion of the implant 9, one end portion of another string 92 is fixed.

The constituent material of the implant 9 is not limited particularly, and various resin materials, fiber and so forth having biocompatibility such as, for example, polypropylene, polyester elastomer or nylon can be used. Further, the constituent material of the strings 91 and 92 is not limited particularly, and various resin materials, fiber and so forth having biocompatibility such as, for example, polypropylene can be used.

Note that the implant 9 is not limited to such a net-like one as described above only if it can exhibit similar effects.

Now, an operation procedure of the puncture apparatus 1, for example, a procedure when the implant 9 is to be embedded into the living body, is described.

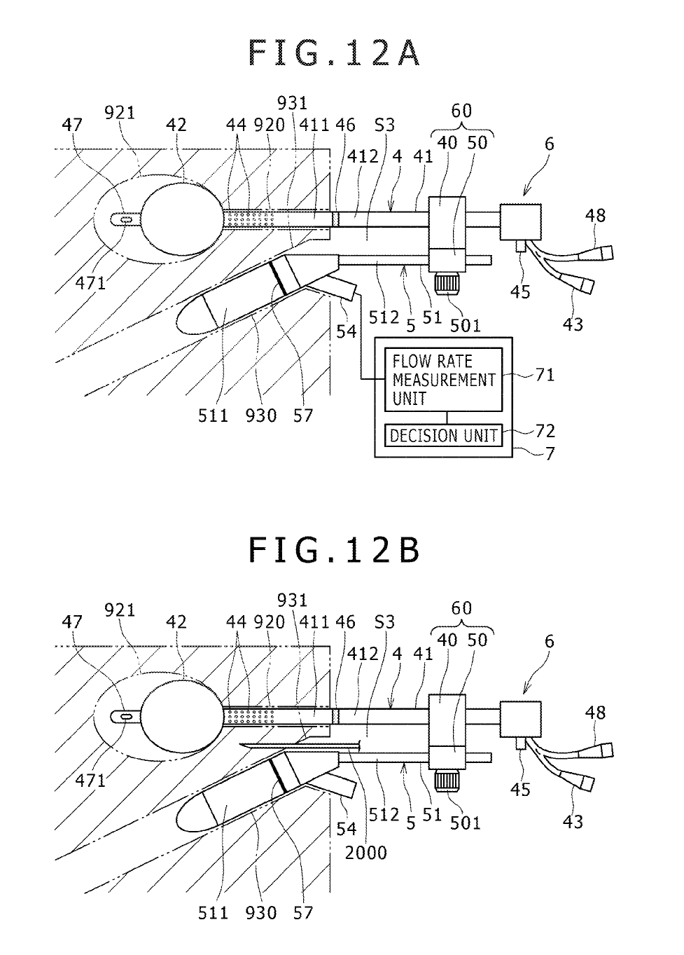

First, a patient is caused to assume a lithotomy position on an operating table, and the insertion tool 6 is mounted on the patient as depicted in FIG. 12A. In accordance with an exemplary embodiment, for example, a urethral insertion portion 41 of the urethral insertion member 4 is inserted into the urethra 920 of the patient. Thereupon, the insertion depth is confirmed from the marker 46 to dispose the balloon 42 in the bladder 921. The urethra 920 is corrected into a predetermined shape by the predetermined shape urethral insertion portion 41. In the present embodiment, the urethra 920 is corrected to a linear state by the urethral insertion portion 41 of a linear shape.

Then, the balloon 42 is inflated and, as occasion demands, the urine is discharged from within the bladder 921 through the discharge hole 471. Further, the vaginal insertion portion 51 of the vaginal insertion member 5 is inserted into the vagina 930 of the patient. Thereupon, the puncture position is confirmed from the marker 57 to insert the vaginal insertion portion 51 to an appropriate depth. Then, the male thread 501 is operated to fix the supporting portions 40 and 50. The mounting of the insertion tool 6 on the patient is completed thereby.

In this state, the non-insertion portions 412 and 512 are spaced away from each other and the supporting portion 60 is spaced away from the body surface between the urethral orifice and the vaginal orifice, and the body surface is exposed as depicted in FIG. 12A. In addition, if the insertion portion 511 and the vaginal front wall 931 are spaced away from each other and a gap (space) is formed between them, a space S3 through which a syringe is to puncture the living body tissue between the urethra 920 and the vagina 930 from the body surface between the urethral orifice and the vaginal orifice is formed.

Then, suction apparatuses are connected to the suction ports 45 and 54 and rendered operative to absorb the urethral rear wall to the urethral insertion portion 41 and absorb the vaginal front wall to the vaginal insertion portion 51. For example, if the urethral rear wall is absorbed precisely to the urethral insertion portion 41, then since the suction hole 44 is closed up with the urethral wall, the suction from the suction port 45 is stopped or weakened. Similarly, if the vaginal front wall is absorbed precisely to the vaginal insertion portion 51, then since the suction hole 53 is closed up with the vaginal wall, the suction from the suction port 54 is stopped or weakened. Therefore, from the absorption degree from the suction portions 45 and 54 (for example, from the magnitude of sound generated by the suction), the operator can confirm whether or not the urethral rear wall and the vaginal front wall are absorbed precisely to the urethral insertion portion 41 and the vaginal insertion portion 51, respectively.

Note that the insertion tool 6 may have a confirmation mechanism 7 for mechanically confirming an absorption state. The confirmation mechanism 7 is not limited particularly if it can confirm an absorption state. However, the confirmation mechanism 7 can be configured such that it can include, for example, as depicted in FIG. 12A, a flow rate measurement unit (negative pressure gauge) 71 for measuring the flow rate from the suction port 54 and a decision unit 72 for deciding on the basis of a result of the measurement from the flow rate measurement unit 71 whether or not absorption is carried out precisely.

Then, humoral peeling off is carried out. In accordance with an exemplary embodiment, for example, the puncture needle of the syringe 2000 is caused to puncture the vaginal front wall 931 through the space (space S3) between the insertion portion 511 and the vaginal front wall 931 as depicted in FIG. 12B, and liquid of saline solution, local anesthetic or the like is injected into the living body tissue between the urethra 920 and the vagina 930 (between the regions S1 and S2). Consequently, the living body tissue between the regions S1 and S2 is expanded, the urethral rear wall is pressed against the urethral insertion portion 41, and the vaginal front wall 931 is pressed against the vaginal insertion portion 51.

Here, preferably the suction from the suction holes 44 and 53 is carried out continuously also during the humoral peeling off. When the urethral rear wall is pressed against the urethral insertion portion 41 by the humoral peeling off, since the urethral rear wall is further absorbed to the urethral insertion portion 41, the suction from the suction port 45 is stopped or weakened. Similarly, when the vaginal front wall 931 is pressed against the vaginal insertion portion 51, it is further absorbed to the vaginal insertion portion 51, and therefore, the suction from the suction port 45 is stopped or weakened. Accordingly, the operator can confirm from the absorption degrees from the suction portions 45 and 54 whether or not the humoral peeling off has been carried out relatively precisely.

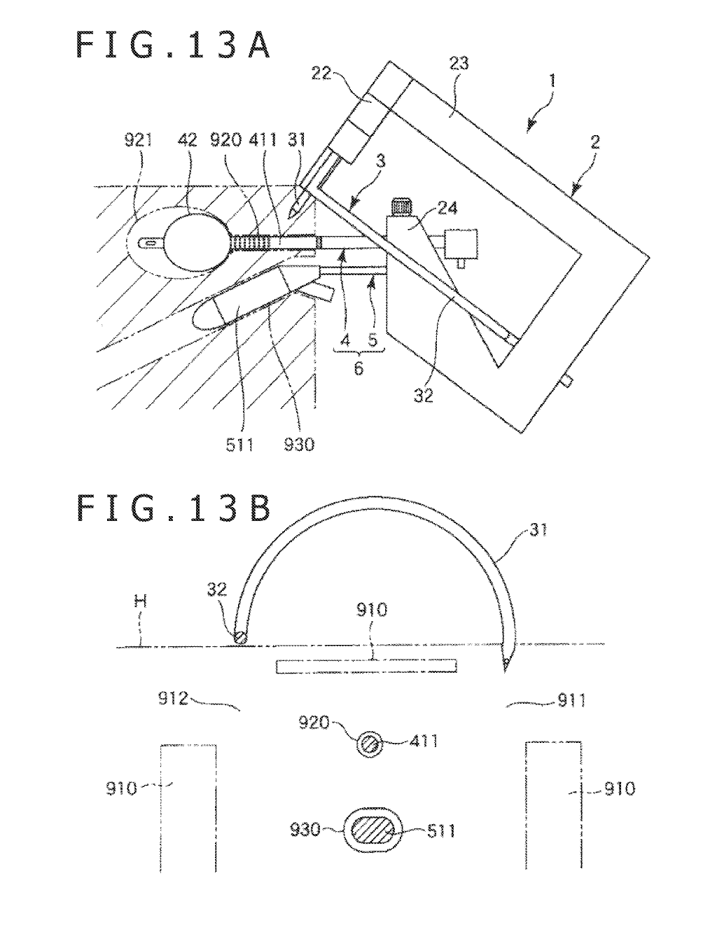

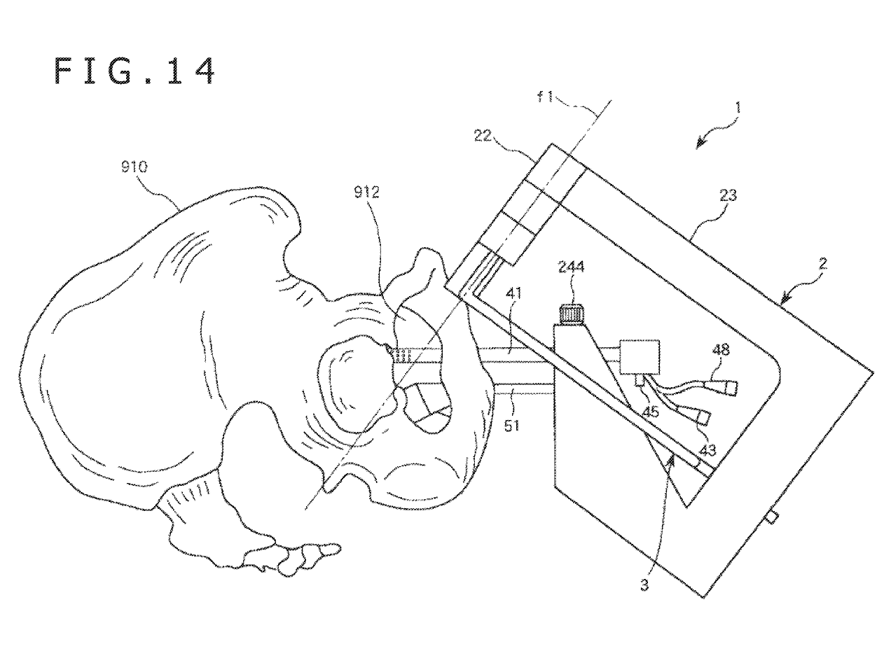

After the humoral peeling off is carried out and the urethral rear wall and the vaginal front wall are placed into a state in which they are spaced away from each other sufficiently, the frame 2 is fixed to the insertion tool 6 as depicted in FIGS. 13A and 13B. Consequently, the puncture apparatus 1 is placed into a state in which it is mounted on the patient. In this state, the positional relationship between the pelvis 910 and the puncture apparatus 1 has such a state as depicted in FIG. 14.

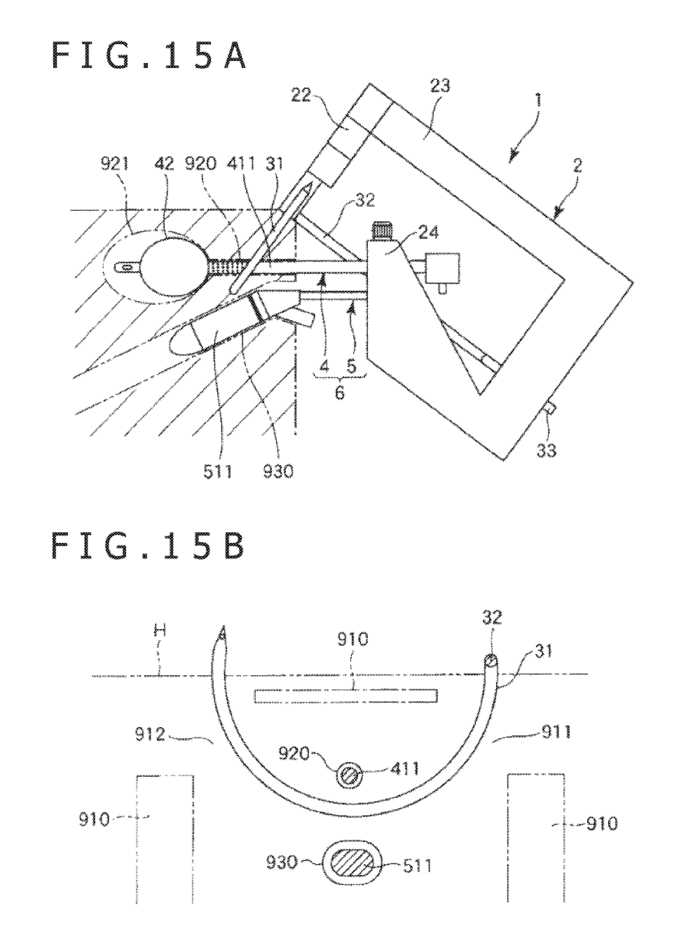

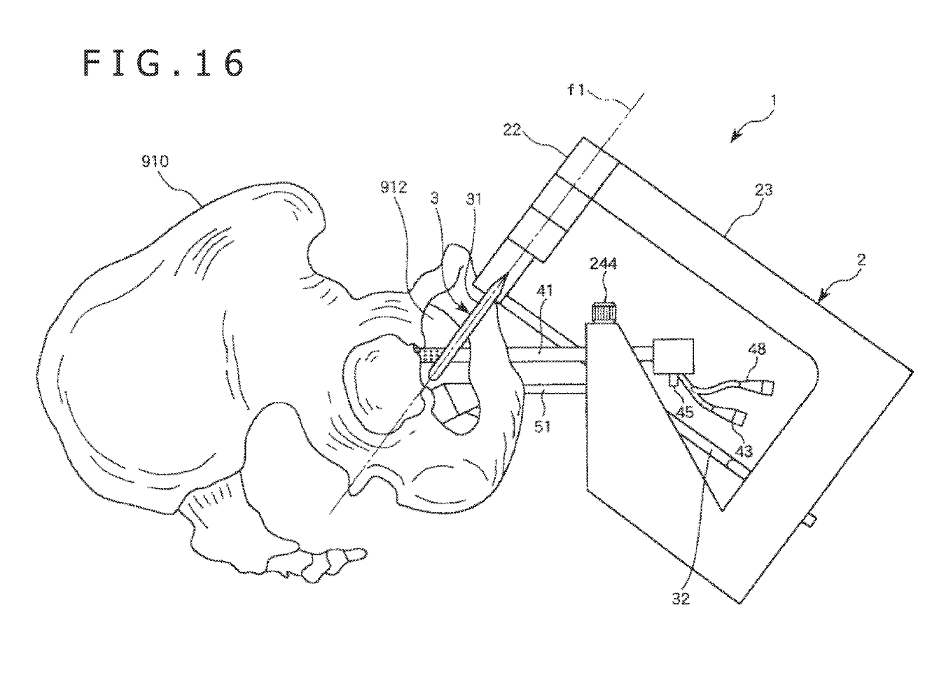

Then, for example, while the connection portion 23 of the frame 2 is gripped by one hand, the puncture member 3 is gripped by the other hand to turn the puncture member 3. Consequently, as depicted in FIG. 15A and 15B, the needle tip of the puncture needle 31 punctures the body surface H of the left side inguinal region or a location in the proximity of the left side inguinal region of the patient and then enters the inside of the body, passes the left side obturator foramen 911, a region between the urethra 920 and the vagina 930 and the right side obturator foramen 912 in order and projects to the outside of the body from the body surface H at the right side inguinal region or a location in the proximity of the right side inguinal region of the patient. In this state, the positional relationship between the pelvis 910 and the puncture apparatus 1 is such as depicted in FIG. 16. In the puncture apparatus 1, since the insertion portion 511 is inclined such that it is spaced away from the insertion portion 411 as described hereinabove, the space between the urethral wall and the vaginal wall can be widen. Therefore, the puncture of the puncture needle 31 described hereinabove can be carried out with a relatively high degree of safety.

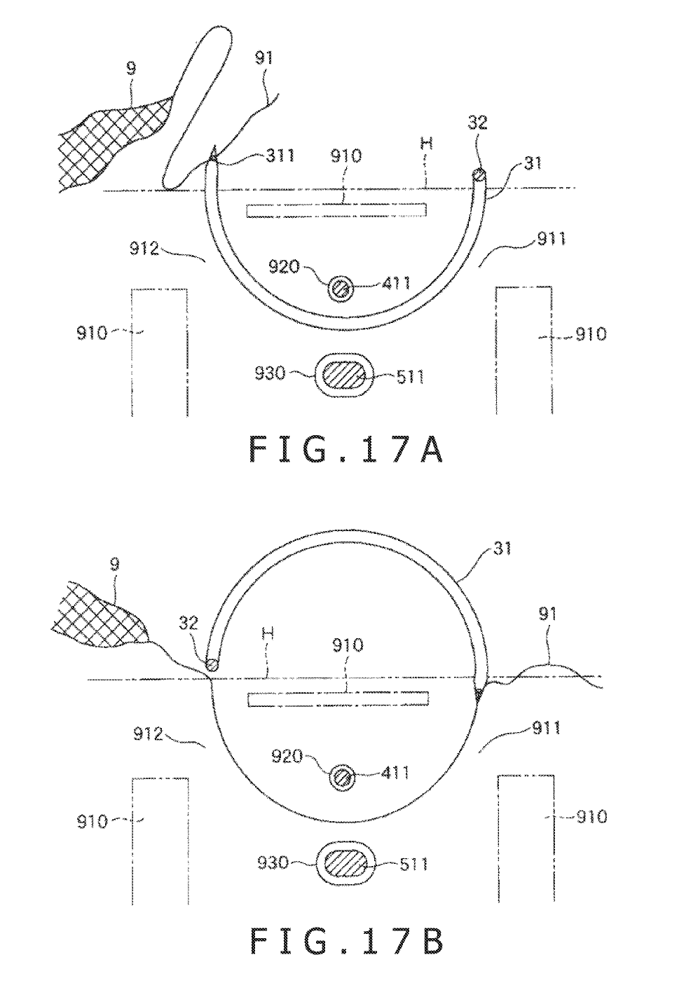

Then, as depicted in FIG. 17A, a string 91 fixed to the implant 9 is threaded into the through-hole 311 of the puncture needle 31 and is held by the puncture needle 31. Then, the puncture member 3 is turned reversely. Consequently, the needle tip of the puncture needle 31 enters the body from the body surface H of the right side inguinal region or a portion in the proximity of the inguinal region of the patient, passes through the obturator foramen 912, the region between the urethra 920 and the vagina 930 and the obturator foramen 911 in order and then goes out from the body surface H in the left side inguinal region or a portion in the proximity of the left side inguinal region of the patent to the outside of the body. In accordance with an exemplary embodiment, the puncture needle 31 is pulled out to the outside of the body. Then, the string 91 is pulled out from the through-hole 311 and the puncture apparatus 1 is removed from the patient. Then, the string 91 is pulled and the implant 9 is indwelled in the living body.

Thereafter, an unnecessary portion of the implant 9 is cut away, thereby ending the manipulation.

As described above, with the insertion tool 6, the positional relationship between the urethral insertion portion 41 (insertion portion 411) and the vaginal insertion portion 51 (insertion portion 511) can be made closer to the positional relationship between the urethra (particularly, the urethra in a rather prolapse state) and the vagina. Therefore, in the mounted state, unnatural deformation and so forth of the urethra 920 or the vagina 930 can be prevented, resulting in reduction of the burden on the patient. Further, since the vaginal wall can be spaced away from the urethral wall along the vaginal insertion portion 511, in the mounted state, the urethral wall and the vaginal wall can be spaced apart from each other. Therefore, with the puncture apparatus 1 of the present disclosure, the puncture permitting region of the puncture needle 31 can be relatively assured to be sufficiently wide, and the contact between the needle tip of the puncture needle 31 and the urethral wall or the vaginal wall can be suppressed effectively and the manipulation can be carried out relatively safely.

In accordance with an exemplary embodiment, since the space S3 can be formed in the mounted state, humoral peeling off can be carried out readily and smoothly. Further, with the puncture apparatus 1, when the implant 9 is to be indwelled, this can be coped with only by a minimally invasive manipulation such as puncture of the puncture needle 31, and highly invasive incision or the like need not be carried out. Therefore, the burden on the patient is relatively light and the safety of the patient is relatively high. Further, the living body can be punctured avoiding the urethra 920 and the vagina 930 by the puncture needle 31, and puncture of the urethra 920 or the vagina 930 by the puncture needle 31 can be performed relatively safely.

Further, such a situation that the implant 9 is exposed to the inside of the vagina from a wound generated by incision as in the case in which the vagina is incised in a conventional manner or such complications that infection is caused through the wound occur can be prevented. This is very safe, and the implant 9 can be embedded with relative certainty.

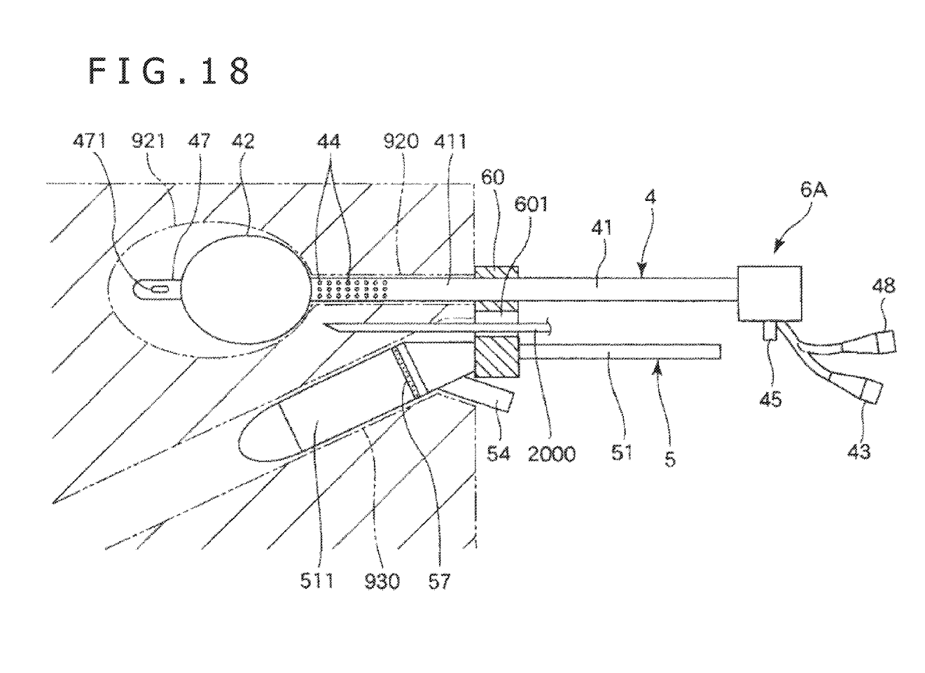

FIG. 18 is a lateral view of an insertion tool, which includes a puncture apparatus according to a second exemplary embodiment of the present disclosure.

In the following, description is given principally of differences of the second embodiment from the first embodiment described hereinabove, and description of similar matters is omitted herein.

As depicted in FIG. 18, the insertion tool 6 which the puncture apparatus of the present embodiment has contacts, when it is mounted on a patient, at the supporting portion 60 thereof with the living body. Further, the supporting portion 60 has formed therein a through-hole 601 for threading a syringe 2000 therein. The insertion tool 6 can carry out humoral peeling off by causing the puncture needle of the syringe 2000 to puncture the living body from between the insertion portion 511 and the vaginal front wall through the through-hole 601.

Also with such a second embodiment as described above, similar effects to those achieved by the first embodiment described hereinabove can be achieved.

Now, a third embodiment of a puncture apparatus of the present disclosure is described.

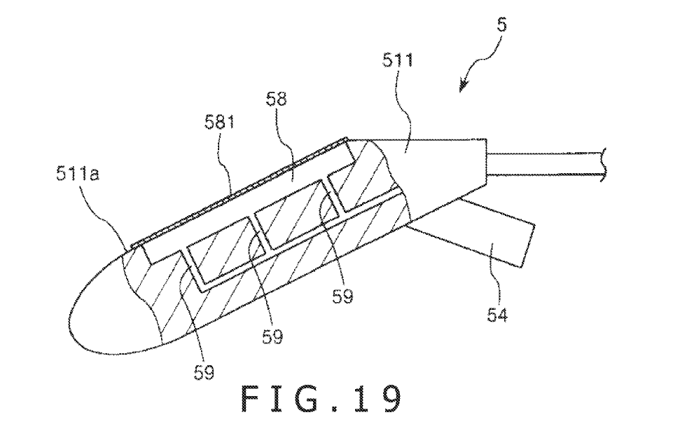

FIG. 19 is a cross sectional view of an insertion tool which includes a puncture apparatus according to the third exemplary embodiment of the present disclosure.

In the following, description is given principally of differences of the third embodiment from the first embodiment described hereinabove, and description of similar matters is omitted herein.

As depicted in FIG. 19, a bottomed recessed portion 58 is provided on the upper face 511a of the insertion portion 511. The recessed portion 58 has a formation region similar to the region S2 described hereinabove in connection with the first embodiment. Further, a plurality of suction holes 59 are formed on a bottom wall of the recessed portion 58. Further, the opening of the recessed portion 58 is covered with a mesh-like body 581 of a mesh or the like. The mesh-like body 581 can be configured, for example, from an article formed by crossing linear objects with each other into a braid (lattice shape), for example, a net-like braid.

Also with such a third embodiment as described above, similar effects to those achieved by the first embodiment described hereinabove can be achieved.

The insertion tool and the puncture apparatus of the present disclosure have been described on the basis of the embodiments depicted in the drawings. However, the present disclosure is not limited to them, and the configuration of the components can be replaced by elements of an arbitrary configuration which have similar functions. Further, the insertion tool and the puncture apparatus may have an arbitrary component added thereto.

Further, while the foregoing description of the embodiments is directed to the configuration by which it can be confirmed on the basis of a suction condition from a suction port whether or not humoral peeling off is carried out precisely, the confirmation may be carried out by some other method. For example, an image pickup element such as a charge coupled device may be disposed in the insertion portion of the vaginal insertion portion such that the confirmation is carried out on the basis of an image obtained from the image pickup device. This similarly applies also to the urethral insertion portion. Alternatively, for example, a lumen into which an endoscope can be inserted may be formed at the vaginal insertion portion such that the confirmation is carried out using the endoscope.

Further, the embodiments described above are directed to a case in which the puncture apparatus of the present disclosure is applied to a device which is used when an implant which can be embedded for the treatment of urinary incontinence of a female is embedded into a living body. However, the application of the puncture apparatus of the present disclosure is not limited to this.

For example, the present disclosure has an application target including excretion failure (such as urinary urgency, frequent urination, urinary incontinence, fecal incontinence, urinary retention and difficulty in urination), pelvic floor disorders including pelvic organ prolapse, vesicovaginal fistula, urethrovaginal fistula, pelvic pain and so forth, which are caused by weakening of the pelvic floor muscles. The pelvic organ prolapse can include such diseases as cystocele, enterocele, rectocele and hysterocele. Alternatively, the pelvic organ prolapse can include such diseases as forward vaginal wall prolapse, rearward vaginal wall prolapse, vaginal apical prolapse and vaginal vault prolapse which are ways to call classified depending upon the prolapsed vaginal wall region.

Further, the hypermobility organizations include the bladder, the vagina, the uterus, intestines and so forth. The fine-moving organizations include bones, muscles, fasciae, ligaments and so forth. For example, in the pelvic floor disorders, obturator fasciae, coccygeus fasciae, cardinal ligaments, uterosacral ligaments, sacrospinous ligaments and so forth are included.

The manipulations for connecting a hypermobility organization to a fine-moving organization in pelvic floor diseases include a retropubic sling surgery, a transobturator sling surgery (transobturator tape (TOT) surgery), a tension-free vaginal mesh (TVM), a uterosacral ligament suspension (USLS), a sacrospinous ligament fixation (SSLF), a iliococcygeus fascia fixation, a coccygeus fascia fixation and so forth.

In accordance with an exemplary embodiment, an insertion tool of the present disclosure can include a urethral insertion portion of a longitudinal shape configured to be inserted into the urethra, and a vaginal insertion portion of a longitudinal shape configured to be inserted into the vagina, and a distal end portion of the vaginal insertion portion is inclined with respect to the urethral insertion portion in a direction in which the distal end portion of the vaginal insertion portion is spaced away from the urethral insertion portion. Therefore, the positional relationship between the urethral insertion portion and the vaginal insertion portion can be made closer to the positional relationship between the urethra and the vagina. As a result, in the mounted state in which the urethral insertion portion is inserted in the urethra and the vaginal insertion portion is inserted in the vagina, unnatural deformation and so forth of the urethra and the vagina can be prevented, resulting in reduction of the burden on the patient. Further, since the vaginal wall can be spaced away from the urethral wall along the vaginal insertion portion, in the mounted state, the urethral wall and the vaginal wall can be spaced widely from each other.

Accordingly, the insertion tool of the present disclosure has industrial applicability.

The detailed description above describes an insertion tool and a puncture apparatus. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An insertion tool, comprising:
a urethral insertion portion of a longitudinal shape configured to be inserted into a urethra; and
a vaginal insertion portion of a longitudinal shape configured to be inserted into a vagina,
wherein a distal end portion of the vaginal insertion portion is inclined with respect to the urethral insertion portion in a direction in which the distal end portion of the vaginal insertion portion is spaced away from the urethral insertion portion.

2. The insertion tool according to claim 1, wherein an extension line of the distal end portion of the vaginal insertion portion and the urethra insertion portion cross with each other.

3. The insertion tool according to claim 1, wherein an extension line of the distal end portion of the vaginal insertion portion and the urethral insertion portion do not cross with each other.

4. The insertion tool according to claim 1, wherein the distal end portion of the vaginal insertion portion is inclined by 10 to 45 degrees with respect to the urethral insertion portion.

5. The insertion tool according to claim 1, wherein the distal end portion of the vaginal insertion portion is spaced away from the urethral insertion portion in an opposite direction in a direction perpendicular to a long axis of the urethral insertion portion.

6. The insertion tool according to claim 1, wherein the distal end portion of the vaginal insertion portion has a flattened shape crushed in an array direction of the urethral insertion portion and the vaginal insertion portion.

7. The insertion tool according to claim 1, wherein at least one suction hole for sucking a portion of a vaginal wall on the urethra side is provided at the distal end portion of the vaginal insertion portion.

8. The insertion tool according to claim 7, wherein a region in which the suction hole is formed has a width from 15 to 35 mm.

9. The insertion tool according to claim 1, comprising:
a urethral non-insertion portion which is not inserted into the urethra, the urethral non-insertion portion being provided on a proximal end side of the urethral insertion portion;
a vaginal non-insertion portion which is not inserted into the vagina, the vaginal non-insertion portion being provided on a proximal end side of the vaginal insertion portion; and
the urethral non-insertion portion and the vaginal non-insertion portion are spaced apart from each other.

10. The insertion tool according to claim 1, comprising
a supporting portion configured to support the urethral insertion portion and the vaginal insertion portion,
wherein the supporting portion is spaced away from a body surface in a mounted state in which the urethral insertion portion is inserted in the urethra and the vaginal insertion portion is inserted in the vagina.

11. A puncture apparatus, comprising:
an insertion tool comprising:
a urethral insertion portion of a longitudinal shape configured to be inserted into a urethra, and
a vaginal insertion portion of a longitudinal shape configured to be inserted into a vagina,
a distal end portion of the vaginal insertion portion being inclined with respect to the urethral insertion portion in a direction in which the distal end portion of the vaginal insertion portion is spaced away from the urethral insertion portion; and
a puncture needle capable of turning with respect to the insertion tool,
wherein, when the puncture needle is turned to puncture a living body tissue, a needle tip of the puncture needle passes between the urethral insertion portion and the vaginal insertion portion.

12. The puncture apparatus according to claim 11, wherein the distal end portion of the vaginal insertion portion is spaced away from the urethral insertion portion in an opposite direction in a direction perpendicular to a long axis of the urethral insertion portion.

13. The puncture apparatus according to claim 11, wherein the distal end portion of the vaginal insertion portion has a flattened shape crushed in an array direction of the urethral insertion portion and the vaginal insertion portion.

14. The puncture apparatus according to claim 11, wherein at least one suction hole for sucking a portion of a vaginal wall on the urethra side is provided at the distal end portion of the vaginal insertion portion.

15. A method of forming a path in living body tissue, the method comprising:
inserting a urethral insertion portion of a longitudinal shape into a urethra;
inserting a vaginal insertion portion of a longitudinal shape into a vagina, wherein a distal end portion of the vaginal insertion portion being inclined with respect to the urethral insertion portion in a direction in which the distal end portion of the vaginal insertion portion is spaced away from the urethral insertion portion; and
puncturing a living body tissue with a puncture needle by passing a needle tip of the puncture needle between the urethral insertion portion and the vaginal insertion portion.

16. The method according to claim 15, wherein the puncture needle is capable of turning with respect to the urethral insertion portion and the vaginal insertion portion

17. The method according to claim 15, wherein the distal end portion of the vaginal insertion portion is spaced away from the urethral insertion portion in an opposite direction in a direction perpendicular to a long axis of the urethral insertion portion.

18. The method according to claim 15, wherein the distal end portion of the vaginal insertion portion has a flattened shape crushed in an array direction of the urethral insertion portion and the vaginal insertion portion.

19. The method according to claim 15, comprising:
sucking a portion of a vaginal wall on the urethra side with at least one suction hole, the at least one suction hole being provided at the distal end portion of the vaginal insertion portion.

20. The method according to claim 15, comprising:
wherein the distal end portion of the vaginal insertion portion is inclined by 10 to 45 degrees with respect to the urethral insertion portion.
